(19) United States
(12) Patent Application Publication
(10) Pub. No.: US 2017/0189059 A1
(43) Pub. Date: Jul. 6, 2017

(54) PERCUTANEOUS ACCESS DEVICE

(71) Applicant: BOSTON SCIENTIFIC SCIMED, INC., Maple Grove, MN (US)

(72) Inventors: Jerry T. Long, JR., Jamaica Plain, MA (US); Timothy P. Harrah, Cambridge, MA (US); Brandon W. Craft, Edgewater, MD (US); Elizabeth A. Stokley, Baltimore, MD (US); Sebastian Koerner, Berlin (DE); Anant Subramaniam, Owings Mills, MD (US)

(21) Appl. No.: 15/399,139

(22) Filed: Jan. 5, 2017

Related U.S. Application Data
(60) Provisional application No. 62/275,611, filed on Jan. 6, 2016.

(51) Int. Cl. A61B 17/34; A61M 29/02; A61B 17/22

(52) U.S. Cl. CPC A61B 17/3403; A61B 17/22; A61B 17/3423; A61M 29/02; A61B 2017/3405; A61B 2017/3425; A61B 2017/00964

(57) ABSTRACT

Medical devices and methods for using medical devices are disclosed. An access device for accessing a target site comprises a tubular member having a proximal portion and a distal portion. The access device may also include a balloon coupled to the tubular member, the balloon extending between the tubular member proximal portion and the tubular member distal portion. The access device may also include a stylet moveable with respect to the tubular member, wherein the stylet is configured to pierce through the skin of a patient.

### CROSS-REFERENCE TO RELATED APPLICATIONS

This application claims priority to U.S. Provisional Patent Application Ser. No. 62/275,611, filed on Jan. 6, 2016, the contents of which are fully incorporated herein by reference.

### TECHNICAL FIELD

The present disclosure pertains to medical devices, and methods for manufacturing medical devices. More particularly, the present disclosure pertains to elongated intracorporeal medical devices for accessing a target site in a human body and methods for manufacturing and using such devices.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device includes an access device for accessing a target site, comprising:

a tubular member having a proximal portion and a distal portion;

a balloon coupled to the tubular member, the balloon extending between the tubular member proximal portion and the tubular member distal portion; and

a stylet moveable with respect to the tubular member, wherein the stylet is configured to pierce through the skin of a patient.

Alternatively or additionally to any of the embodiments above, wherein the stylet includes a tip having a distal tip portion and a proximal tip portion, and wherein the tip tapers from the proximal tip portion to the distal tip portion.

Alternatively or additionally to any of the embodiments above, wherein the tubular member includes at least one closed end.

Alternatively or additionally to any of the embodiments above, wherein the tubular member includes a lumen extending therethrough.

Alternatively or additionally to any of the embodiments above, wherein at least a portion of the stylet extends within at least a portion of the lumen of the tubular member.

Alternatively or additionally to any of the embodiments above, further comprising a marker, wherein the marker is configured to determine the distance between the access device and the skin of the patient.

Alternatively or additionally to any of the embodiments above, wherein the marker includes a radiopaque portion.

Alternatively or additionally to any of the embodiments above, wherein the marker includes an echogenic portion.

Alternatively or additionally to any of the embodiments above, further comprising a balloon inflation port, wherein the balloon inflation port is coupled to the proximal portion of the tubular member, a proximal portion of the balloon, or both.

Alternatively or additionally to any of the embodiments above, further comprising a metal scaffold, and wherein the metal scaffold is designed to be deployed by the balloon.

An example method for manufacturing a medical device includes accessing a target site in a body, the method comprising:

piercing the skin of a patient with an access device to create an access opening, the access device including a tubular member having a dilatation balloon coupled thereto and a stylet removably coupled to the tubular member;

advancing the access device toward a target site;

determining the position of the access device in relation to the patient's skin;

expanding the dilatation balloon adjacent the access opening such that the access opening is enlarged; and

withdrawing the access device from the patient's body.

Alternatively or additionally to any of the embodiments above, wherein the tubular member includes a lumen extending therein, and wherein the stylet extends within at least a portion of the lumen of the tubular member.

Alternatively or additionally to any of the embodiments above, wherein the access device further comprises a marker, and wherein the marker is configure to determine the distance between the access device and the patient's skin.

Alternatively or additionally to any of the embodiments above, further comprising advancing an access sheath through the access opening prior to withdrawing the access device from the patient's body.

Alternatively or additionally to any of the embodiments above further comprising a stent coupled to dilatation balloon.

Alternatively or additionally to any of the embodiments above, wherein expanding the dilatation balloon adjacent the access opening further comprises deploying the stent through the access opening.

An example system for removing a kidney stone from the body is disclosed, the system comprising:

an access needle including a tubular member having a proximal portion, a distal portion and a lumen extending therein, a balloon coupled to the tubular member and a stylet positioned within the lumen of the tubular member, the stylet configured to pierce through the skin of a patient; and

a sheath including a lumen extending therein, wherein the sheath is configured to maintain an access pathway through the skin of the patient.

Alternatively or additionally to any of the embodiments above, wherein the sheath includes at least one marker, wherein the marker is configured measure the distance that the sheath is advanced into the patient.

Alternatively or additionally to any of the embodiments above, wherein the sheath is configured to be advanced over the access needle and through the skin of the patient.

Alternatively or additionally to any of the embodiments above, wherein the sheath includes a metal scaffold, and wherein the metal scaffold is designed to be deployed by the balloon.

Alternatively or additionally to any of the embodiments above, wherein the metal scaffold includes a pull member positioned on a proximal portion of the stent.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description of various embodiments of the disclosure in connection with the accompanying drawings, in which:

FIG. 1 is a partial cross-sectional view of an example medical device accessing a target site.

FIG. 2 is a partial cross-sectional view of an example medical device.

FIG. 3 is a partial cross-sectional view of another example medical device including a stylet.

FIG. 4 is a partial cross-sectional view of another example medical device including a balloon member and a stylet.

FIG. 5 is a partial cross-sectional view of another example medical device including a balloon member and a stylet.

FIG. 6 is a partial cross-sectional view of another example medical device including a balloon member and a stylet positioned in an access sheath.

FIG. 7 is a cross-sectional view of an example access sheath.

FIG. 8 is a partial cross-sectional view of another example medical device including a balloon member, and expandable member and a stylet.

FIG. 9 is a cross-sectional view of an example expandable member.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Treatment of kidney stones may be accomplished through a variety of methodologies. For example, the removal of kidney stones may include the application of a minimally invasive therapy (e.g., laser therapy, shock wave therapy) to break the stones into pieces small enough to pass spontaneously out of the body. However, in some instances kidney stone removal may require more invasive therapies. For example, percutaneous nephrolithotomy may be performed to surgically remove a kidney stone. Percutaneous nephrolithotomy may require puncturing the skin and placing a hollow tube through that puncture site to access a kidney. The hollow tube may then be used to position a probe necessary to remove the stone. However, in some instances placement of the hollow tube may require serial dilation of the initial puncture site.

For example, the initial puncturing of the skin may be accomplished via a small stylet/cannula, through which a guidewire may extend. The guidewire may be utilized to exchange numerous devices designed to serially dilate an access tract large enough to accommodate the hollow tube. Therefore, in some instances it may be desirable to utilize materials and/or design an access device that reduces the number of medical instruments necessary to create the access tract. Some of the examples and methods disclosed herein may include a percutaneous access device that can reduce the number of devices utilized to create a percutaneous access tract.

The access devices disclosed herein may treat kidney stones. Additionally, the access devices disclosed may utilized to treat other forms of disease, including gastrointestinal, airway, urethra, ureter, cardiac, brain, breast, bladder, and peripheral vascular disease, for example. Further, the percutaneous access devices disclosed herein may also be used to access numerous body cavities having both solid and/or hollow organs.

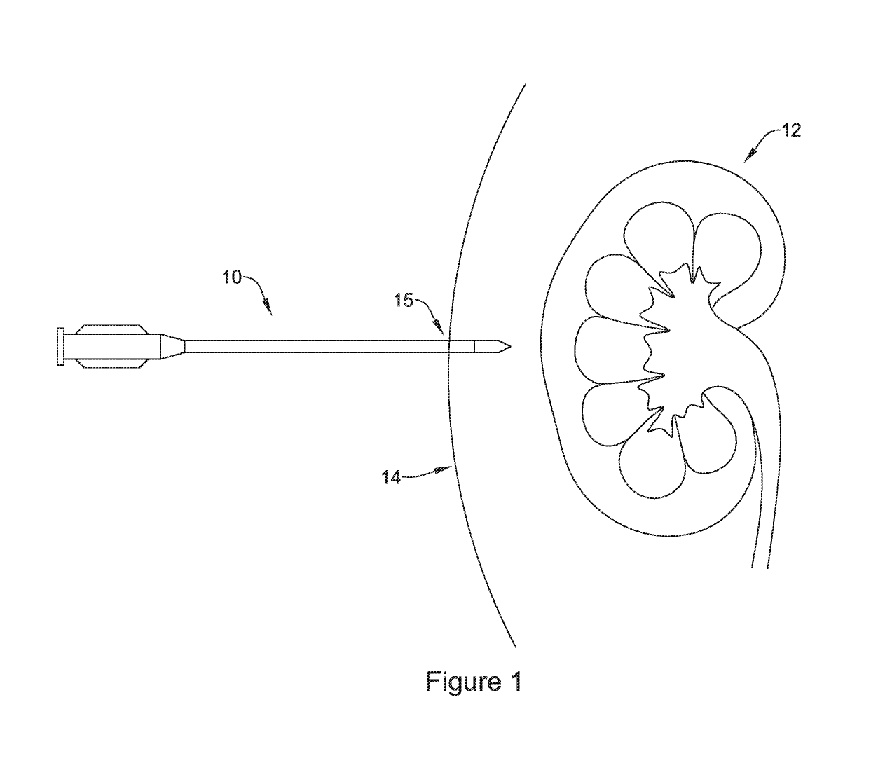

FIG. 1 shows an example access device system 10. As shown in FIG. 1, access device 10 may be utilized to create a percutaneous passageway, conduit and/or access tract to a target organ 12. For example, FIG. 1 shows access device 10 extending through the skin 14 of an example patient. Further, access device 10 may extend through the skin 14 of a patient via an access site (e.g., opening) 15. The access site 15 may be defined as a surgical incision made in the skin 14 to gain access into the body cavity. Further, FIG. 1 depicts a target organ 12. As shown in FIG. 1, target organ 12 may be a kidney, however, it is contemplated that access device 10 may be utilized to access other organs and/or areas of the body.

As discussed above, in some instances access system 10 may be used to create an access tract to a body organ (e.g., kidney). The access tract may be utilized to position a hollow tube within close proximity of the target organ. Further, the tubular member may be designed to permit and/or accommodate larger instruments therethrough.

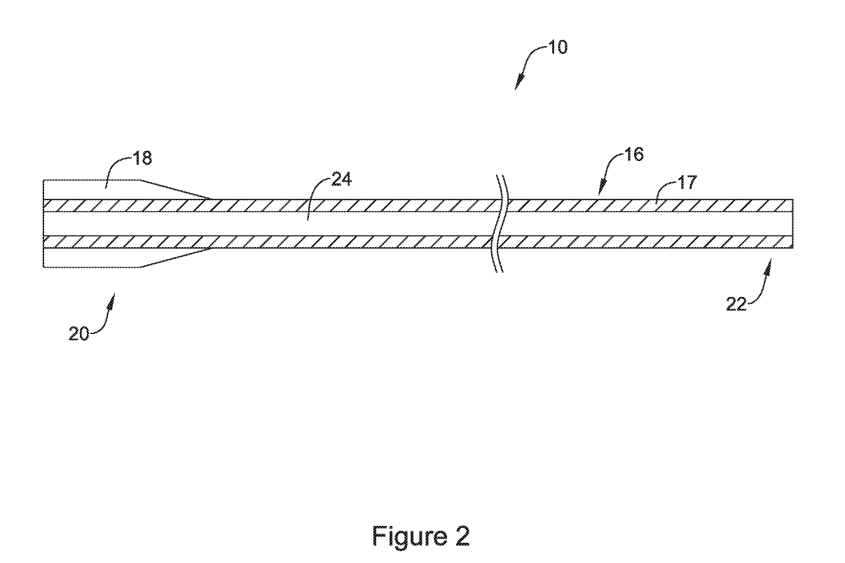

Positioning the hollow tube within the body cavity may require opening and/or dilating puncture site 15 after an initial incision is created. In some instances, opening and/or dilating puncture site 15 may initially begin with the insertion of a stylet and/or tubular member through an access site 15. For example, FIG. 2 shows access system 10 including a tubular member 16. Tubular member 16 may include a proximal portion 20 and a distal portion 22. Further, in some instances, tubular member 16 may include a lumen 24 extending therein. In some instances, lumen 24 may extend the entire length of tubular member 16. In other instances lumen 24 may extend only a portion of the length of tubular member 16. For example, in some instances tubular member 16 may include a closed end.

In some instances, tubular member 16 may be coupled to a manifold 18. For example, as shown in FIG. 2, manifold 18 may be coupled to a proximal portion of tubular member 16. Manifold 18 may be utilized as a handle and/or the gripping portion. For example, when tubular member 16 is placed through an access site 15, a clinician may grip and/or hold on to manifold 18 while manipulating the position and or insertion depth of tubular member 16.

Tubular member 16 may be constructed using a variety of manufacturing techniques and/or designs. For example, tubular member 16 may include one or more layers including various materials. Further, tubular member 16 may include a braided, woven, or knitted structure. Additionally, tubular member 16 may include one or more of slots, cuts, slits, etc. designed to change the performance characteristics (e.g., flexibility) of tubular member 16. While not shown in FIG. 2, tubular member 16 may combine one or more of the design characteristics to customize the performance of tubular member 16.

Tubular member 16 may be generally cylindrical in shape as shown in FIG. 2. However, the shape of tubular member 16 shown in FIG. 2 is not intended to be limiting. Rather, tubular member 16 may include a variety of shapes. For example, while FIG. 2 shows the tubular walls 17 of tubular member 16 being of substantially uniform thickness along the length of tubular member 16, it is contemplated that the tubular wall 17 thickness may vary in any number of configurations along the length of tubular member 16. For example, it is contemplated that the distal portion 22 of tubular member 16 may include a tapered region (not shown). For example, the thickness of one or more of tubular walls 17 may decrease from proximal portion 20 to distal portion 22. In some examples, the thickness of tubular walls 17 may decrease near the distal end of tubular member 16. In other words, tubular member 16 may include a tapered tip.

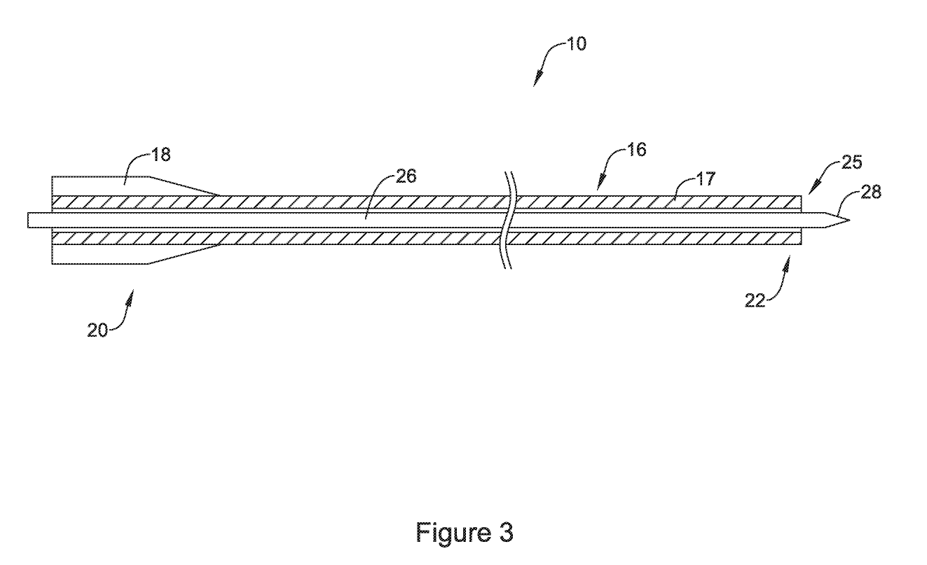

In some instances, the tubular member 16 may be coupled and/or combined with a stylet, needle, or other similar device used to pierce the skin of a patient. For example, FIG. 3 shows access system 10 including tubular member 16 coupled to stylet 26. As shown stylet 26 may be positioned within a portion or along the entire length of lumen 24 or tubular member 16.

Stylet 26 may be defined as a solid member having a tapered and/or pointed tip as illustrated in FIG. 3. However, this is not intended to be limiting. Stylet 26 may include one or more lumens extending along a portion or the entire length of stylet 26. Furthermore, while FIG. 3 shows the tip 28 of the stylet 26 positioned near a distal end of the stylet 26, it is additionally contemplated that the tapered portion of tip 28 may extend along any portion stylet 26. For example, stylet 26 may include a taper along its entire length (or, alternatively, along any portion of its length).

Stylet 26 may be moveable relative to tubular member 16. For example, FIG. 3 shows stylet 26 positioned within lumen 24 of tubular member 16. It is contemplated that stylet 26 may be inserted into a proximal portion 20 of tubular member 16 and extend to a distal portion 22 of tubular member 16. For example, in some examples, stylet 26 may be inserted through manifold 18 and extend out of the distal end 25 of tubular member 16.

In some instances, tubular member 16 and stylet 26 may be utilized in combination to pierce the skin of a patient in order to provide access to a body cavity, organ, etc. For example, in some instances, stylet 26 may be combined, attached, coupled, affixed, releaseably attached and/or removeably attached to tubular member 16 prior to insertion into a patient. For example, tubular member 16 and stylet 16 may be configured as shown in FIG. 3 prior to insertion into a patient.

As shown in FIG. 3, it may be desirable to have the tapered (e.g., sharp) tip 28 of stylet 26 extend from the distal end 25 of tubular member 16 prior to insertion into a patient. For example, the tapered tip 28 of stylet 26 may easily pierce the skin of a patient while minimizing trauma to the surrounding tissue. After stylet 26 creates an initial puncture, tubular member 16 may be more easily advanced through the access site 15.

While the above example discloses the tubular member 16 and stylet 26 being inserted through the skin of a patient in tandem (e.g, coupled together), it is contemplated that the tubular member 16 and stylet 26 may be inserted through the skin of patient separately. For example, it is contemplated that the stylet 26 may be inserted through the skin (e.g., inserted to create an initial puncture site) followed by the tubular member 16.

In some instances, after the tubular member 16 and stylet 26 have been inserted through the skin of a patient, the stylet 26 may be removed from the tubular member 16. Further, removal of stylet 26 may provide an access tract for another medical device to be advanced through tubular member 16. For example, in some instances a guidewire may be advanced through lumen 24 of tubular member 16 after stylet has been removed.

However, in other examples, stylet 26 may remain coupled to tubular member 16 after the puncture of access site 15. Further, in some instances it may be desirable to further dilate access site 15 after tubular member 16 (with or without stylet 26) is positioned across, within, along, and/or through an access site 15.

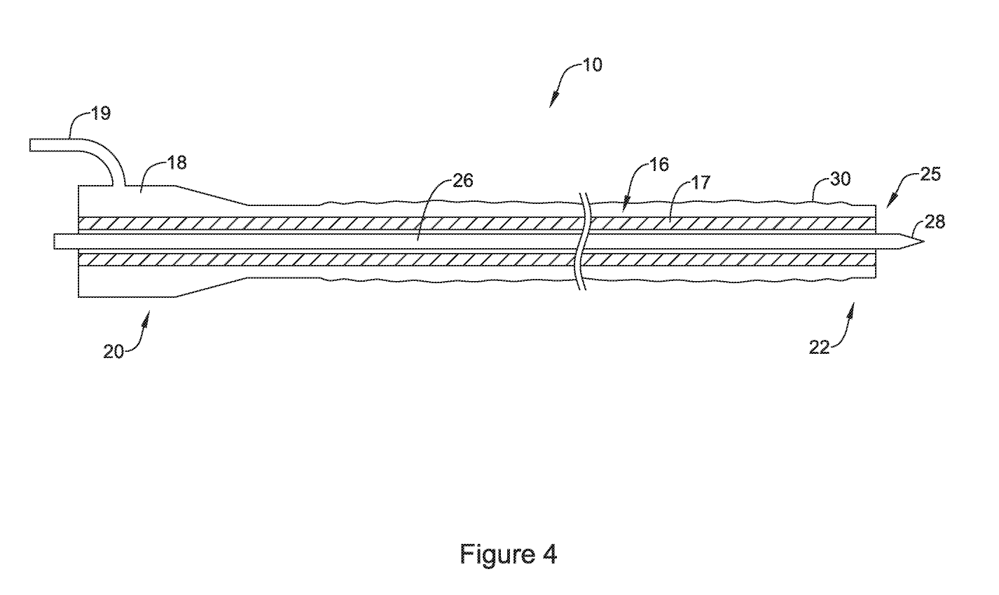

FIG. 4 shows another example medical access device system 10. Similar to that discussed with respect to FIGS. 1-3, access device 10 includes a tubular member 16 coupled to a manifold 18 at a proximal end 20. Further, access device 10 includes a stylet 26 positioned within lumen 24 of tubular member 16. As illustrated in FIG. 4, stylet 26 may extend the entire length of tubular member 16. Further, stylet 26 may extend out of and away from manifold 18 and the distal end 25 of tubular member 16.

Additionally, FIG. 4 shows the tubular member 16 of access device 10 including an inflatable member 30 coupled to the outer surface of tubular member 16. In some examples, inflatable member 30 may be defined as a balloon. As shown in FIG. 4, balloon 30 may extend substantially along the entire length of tubular member 16. However, while shown extending along substantially the entire length of tubular member 16, this is not intended to be limiting. Rather, balloon 30 may extend along only a portion of tubular member 16.

In some examples, inflatable member 30 may be in fluid communication with manifold 18. Further, in some examples, manifold 18 may include an inflation lumen 19. Inflation lumen 19 may be coupled to an inflation device. The inflation device may be utilized to expand balloon member 30.

It can be appreciated that expansion of balloon member 30 (while the access system 10 is positioned across, within, along, and/or through an access site 15) may expand (e.g., dilate, enlarge, etc.) the access site 15. In other words, if the access site 15 is defined as an incision or puncture of a patient's skin, expansion of inflatable member 30 may enlarge and/or expand the patient's skin to a desired diameter.

In addition, in some instances access system 10 (including inflatable member 30) may utilized in combination to both puncture the skin of a patient to create and access site 15 and, thereafter, dilate that access site 15. For example, the access system 10 shown in FIG. 4 may be utilized to puncture the skin of patient using stylet 26. The access system 10 may then be advanced through the skin of the patient to a desired depth. Upon reaching the desired depth into the patient's body, the inflatable member 30 may be expanded to create a desired access tract.

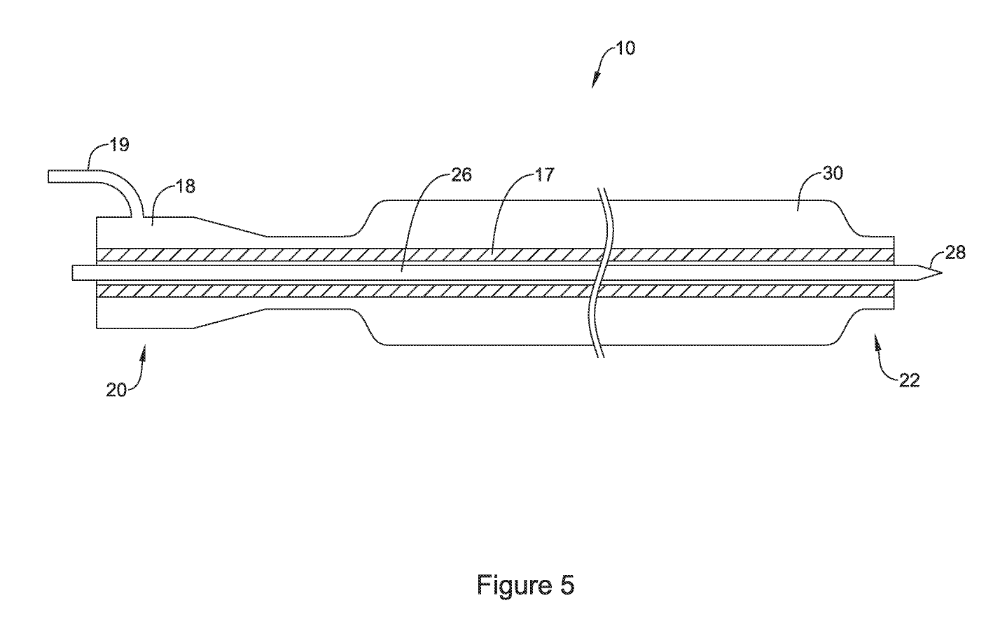

FIG. 5 shows the access system described with respect to FIG. 4. However, FIG. 5 further illustrates the expandable member 30 in the expanded configuration. As shown in FIG. 5, the expandable member 30 may expand radially to an extent 1, 2, 3, 4, 5, 6, 7, 8, 9, 10, 15, 20 or more times its diameter in the unexpanded state.

In some instances it may be desirable to advance additional medical devices through an access site 15. For example, after the inflatable member 30 has been inflated as described above with respect to FIG. 5, it may be desirable to preserve the opening (e.g., the diameter or radial extent) created via expansion of the balloon member 30 within the access site 15.

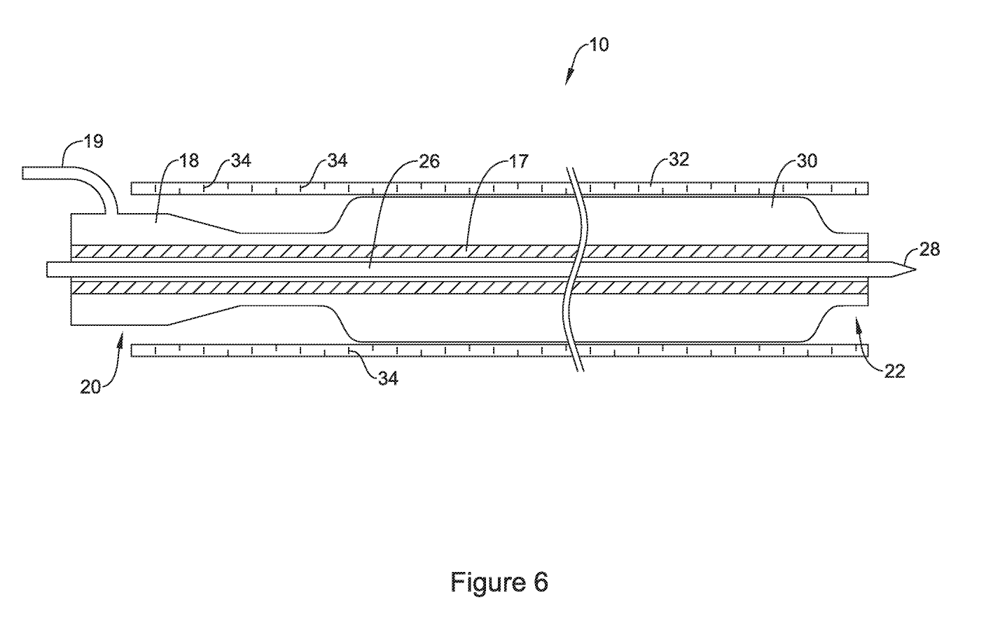

FIG. 6 shows one example of using an additional medical device to maintain the access site opening created via the access system(s) described above. As shown in FIG. 6, in some instances a sheath member 32 may be advanced over the expanded balloon member 30 (in addition to the manifold 18 and proximal/distal portions of balloon member 30). While not shown in FIG. 6, it can be appreciated that sheath member 32 may be advanced from a positioned outside the body, through the skin of the patient (via the access site 15 created by the balloon member 30) to a position inside the body of the patient.

In some examples, sheath member 32 may be a rigid or semi-rigid tubular structure designed to maintain the access site 15 opening created by the access systems 10 described above. Further, similar to that described above with respect to tubular member 16, sheath member 32 may be constructed from a variety of materials and contemplate a variety of design configurations. For example, sheath member 32 may include one or more layers, braids, tapers, slots, slits, etc. Additionally, sheath member 32 may be configured in a variety of lengths, depending on the particular target site the sheath member is being used to access.

In some instances, sheath member 32 may include one or more depth markings 34. While not intended to be limiting, depth markings, in some instances, may be resemble hash marks placed on the outer surface of sheath member 32. In some examples the depth marks may be configured to provide a user with an indication of the distance for which sheath member 32 has been advanced into the body. For example, depth marks 34 may provide an indication for how far the distal end of sheath member 32 has been advanced beyond the access cite 15 (e.g., the patient's skin).

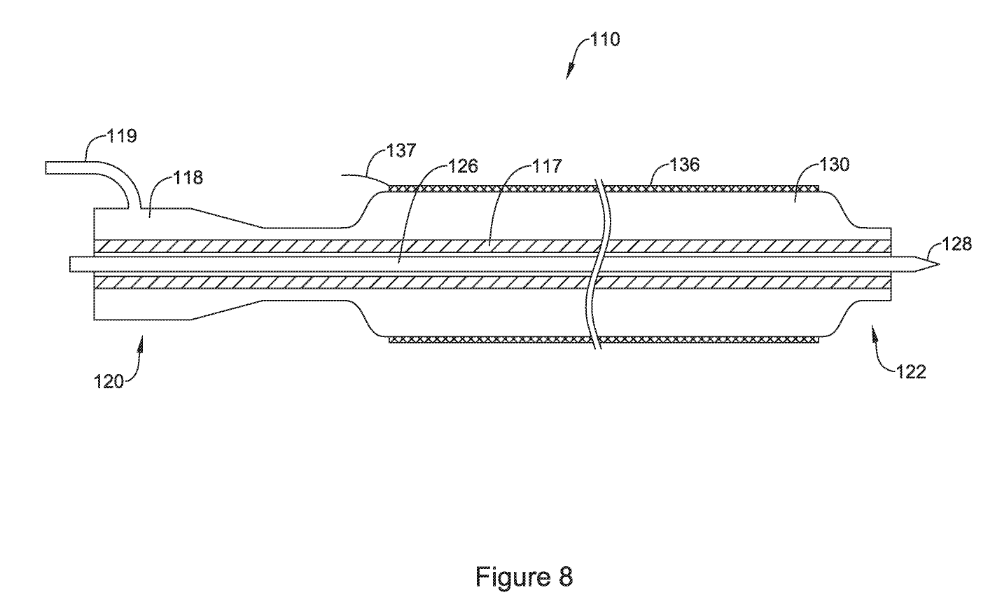

In some examples, it may be desirable to remove the access system 10 after placement of the sheath member 32. FIG. 8 illustrates sheath member 32 after access system 10 has been removed from sheath member 32. It can be appreciated that removal of access system 10 from sheath member 32 may permit larger medical devices to pass through access site 15 via sheath member 32. For example, as can be seen in the figures, sheath member 32 may include an inner lumen that is larger than the inner lumen 24 of tubular member 16, for example.

FIG. 8 shows another example access device 110. Similar to that described above with respect to FIGS. 1-5, access device 110 may include a tubular member 116 coupled, affixed, releasably attached, etc. to a stylet 126. Additionally, access device 110 may include an expandable scaffold 136 coupled to the outer surface of expandable member 130.

In some instances, expandable scaffold 136 may be defined as an expandable stent. Expandable scaffold 136 may be constructed from a variety of materials (e.g., metal, polymer, biodegradable, bioabsorbable, etc.) Further, expandable scaffold 136 may include both self-expanding and balloon expandable stents. Additionally, as shown in FIG. 8, expandable member 136 may extend along a portion or the entire length of balloon member 130.

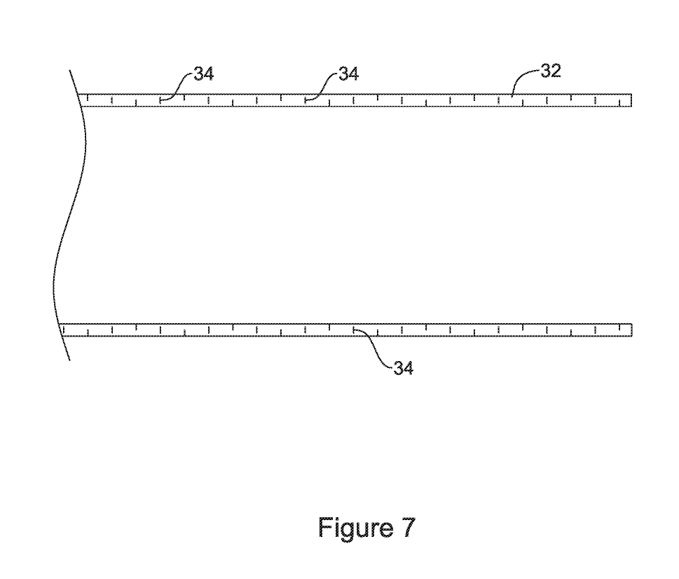

Further, expandable scaffold 136 may provide an access conduit similar to that described above with respect to sheath member 32 shown in FIGS. 6 and 7. For example, while in a collapsed stated, expandable scaffold 136 may be inserted into access site 15 while coupled to tubular member 116 and stylet 126. For example, stylet 126 may be utilized to puncture the skin of a patient to create an access site. Once the access system 110 has been advanced through the access site, balloon member 130 (along with the stylet 126 and/or expandable member 136) may be utilized to dilate the access site 15 to a desired diameter.

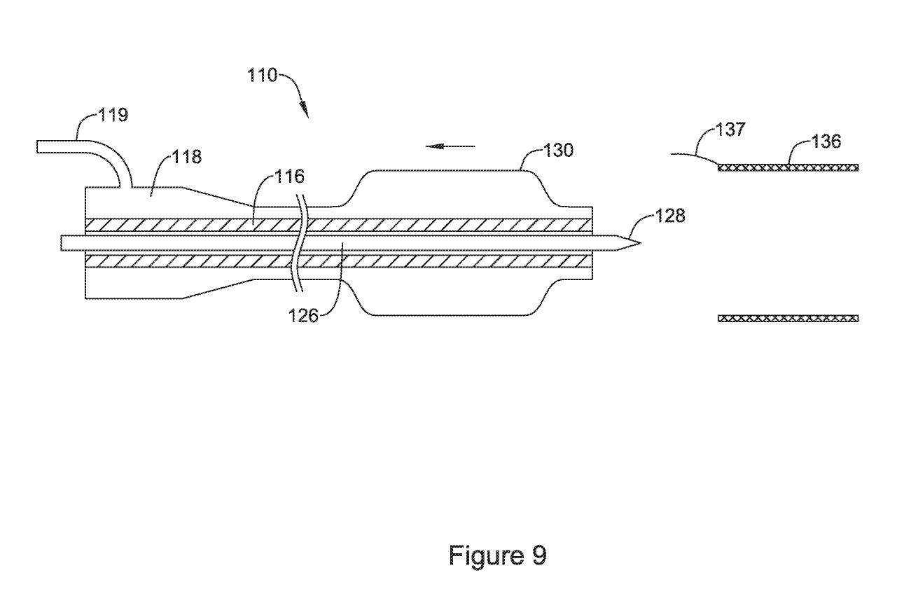

Additionally, once the access site has been dilated to a desired diameter, the access system 110 may be removed, thereby leaving expandable scaffold 136 in place. For example, FIG. 9 shows that after the access site has been dilated to a desired diameter, balloon member 130 may be deflated and access system 110 removed (as indicated by the arrow in FIG. 9). Further, FIG. 9 shows expandable member 136 remaining in an access site (not shown). In a similar manner to that described above with respect to sheath member 32, expandable member 136 may permit larger medical devices to pass through access site.

In some examples, expandable scaffold 136 may include a tab member 137. Tab member 137 may be positioned on a proximal portion of expandable scaffold 136. Tab member 137 may facilitate removal of expandable scaffold 136 from example access site 15. For example, in some instances a clinician may be able to grip and pull tab member 137 in a proximal direction. Pulling tab member 137 may, in turn, cause expandable member 136 to collapse and be removed from an access site. It is contemplated that tab member 137 may be positioned along any portion of expandable member 136.

Further, any of the above examples may include one or more markers to determine the distance between the access device and the skin of the patient. For example, in some instances the tubular member, stylet, balloon structure, sheath, expandable scaffold, etc. may include a marker which may be utilized to determine the distance between the marker and the skin of the patient. For example, in some instances the marker may include a radiopaque and/or echogenic element. Further, the marker may be place on any portion of the access device (e.g., a marker placed on the distal tip of the stylet and/or along the distal portion of the tubular member).

Furthermore, in some examples above it may be desirable to design sheath member 32 and/or expandable scaffold 136 to accommodate particular medical devices therethrough. For example, in some instances sheath member 32 and/or expandable scaffold 136 may be sized to accommodate a range of medical devices including nephroscopes, ureteroscopes or the like. Therefore, in some instances sheath member 32 and/or expandable scaffold 136 may include an inner lumen diameter that is approximately 5-35 Fr. or more (e.g., 5-10 Fr. or more, 5-15 Fr. or more, 10-20 Fr. or more, 15-25 Fr. or more, 20-30 Fr. or more, 25-35 Fr. or more).

Access system 10 and/or other components of access system 10 may be made from a metal, metal alloy, polymer (some examples of which are disclosed below), a metal-polymer composite, ceramics, combinations thereof, and the like, or other suitable material.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

Access system 10 and/or other components of access system 10 may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An access device for accessing a target site, comprising:
a tubular member having a proximal portion and a distal portion;
a balloon coupled to the tubular member, the balloon extending between the tubular member proximal portion and the tubular member distal portion; and
a stylet moveable with respect to the tubular member, wherein the stylet is configured to pierce through the skin of a patient.

2. The access device of claim 1, wherein the stylet includes a tip having a distal tip portion and a proximal tip portion, and wherein the tip tapers from the proximal tip portion to the distal tip portion.

3. The access device of claim 1, wherein the tubular member includes at least one closed end.

4. The access device of claim 1, wherein the tubular member includes a lumen extending therethrough.

5. The access device of claim 4, wherein at least a portion of the stylet extends within at least a portion of the lumen of the tubular member.

6. The access device of claim 1, further comprising a marker, wherein the marker is configured to determine the distance between the access device and the skin of the patient.

7. The access device of claim 6, wherein the marker includes a radiopaque portion.

8. The access device of claim 6, wherein the marker includes an echogenic portion.

9. The access device of claim 1, further comprising a balloon inflation port, wherein the balloon inflation port is coupled to the proximal portion of the tubular member, a proximal portion of the balloon, or both.

10. A method of accessing a target site in a body, the method comprising:
piercing the skin of a patient with an access device to create an access opening, the access device including a tubular member having a dilatation balloon coupled thereto and a stylet removably coupled to the tubular member;
advancing the access device toward a target site;
determining the position of the access device in relation to the patient's skin;
expanding the dilatation balloon adjacent the access opening such that the access opening is enlarged; and
withdrawing the access device from the patient's body.

11. The method of claim 10, wherein the tubular member includes a lumen extending therein, and wherein the stylet extends within at least a portion of the lumen of the tubular member.

12. The method of claim 10, wherein the access device further comprises a marker, and wherein the marker is configure to determine the distance between the access device and the patient's skin.

13. The method of claim 10, further comprising advancing an access sheath through the access opening prior to withdrawing the access device from the patient's body.

14. The method of claim 10, further comprising a stent coupled to dilatation balloon.

15. The method of claim 14, wherein expanding the dilatation balloon adjacent the access opening further comprises deploying the stent through the access opening.

16. A system for removing a kidney stone from the body, the system comprising:
an access needle including a tubular member having a proximal portion, a distal portion and a lumen extending therein, a balloon coupled to the tubular member and a stylet positioned within the lumen of the tubular member, the stylet configured to pierce through the skin of a patient; and
a sheath including a lumen extending therein, wherein the sheath is configured to maintain an access pathway through the skin of the patient.

17. The system of claim 16, wherein the sheath includes at least one marker, wherein the marker is configured measure the distance that the sheath is advanced into the patient.

18. The system of claim 16, wherein the sheath is configured to be advanced over the access needle and through the skin of the patient.

19. The system of claim 16, wherein the sheath includes a metal scaffold, and wherein the metal scaffold is designed to be deployed by the balloon.

20. The system of claim 19, wherein the metal scaffold includes a pull member positioned on a proximal portion of the stent.
